(19) United States
(12) Patent Application Publication
(10) Pub. No.: US 2024/0165373 A1
(43) Pub. Date: May 23, 2024

(54) WETTED MEDICAL DEVICES AND METHODS FOR IMPLANTING WETTED MEDICAL DEVICES IN PATIENTS

(71) Applicant: Freeflow Medical Devices LLC, Lancaster, PA (US)

(72) Inventors: Saibal Bandyopadhyay, Lancaster, PA (US); Andrew K. Jones, Lancaster, PA (US)

(21) Appl. No.: 18/512,868

(22) Filed: Nov. 17, 2023

Related U.S. Application Data
(60) Provisional application No. 63/426,347, filed on Nov. 17, 2022.

(51) Int. Cl. A61M 25/00; A61L 29/14; A61L 31/14; A61M 27/00

(52) U.S. Cl. CPC A61M 25/0045; A61L 29/14; A61L 31/14; A61M 27/006; A61L 2300/202; A61L 2400/10; A61M 2025/0046; A61M 2205/0222

(57) ABSTRACT

Disclosed herein is a wetted medical device, methods for preparing wetted medical devices, and kits to make the same. The disclosed wetted medical device includes a self-healing layer of lubricating fluid on a surface of the medical device. The surface includes a polymer layer having a roughened texture, which interacts with and attracts the lubricating fluid to form a lubricating fluid layer.

### CROSS-REFERENCE TO RELATED APPLICATIONS

This application claims the benefit of priority to U.S. Provisional Application No. 63/426,347, filed Nov. 17, 2022, which is incorporated herein by reference in its entirety.

### TECHNICAL FIELD

The disclosure relates to wetted medical devices and method for preparing wetted medical devices.

### BACKGROUND

Certain medical devices require a surface which is wet with a specific liquid when the device is implanted in a patient. New biomaterials combine porous and non-porous surfaces on which a specific liquid is applied which improves the device performance in ways such as reducing clotting, reducing clogging, improving reendothelialization of surfaces, and reducing encapsulation.

These implanted devices frequently take time to implant, with some procedures requiring more than an hour to perform. The implanted devices must be fully wetted when the procedure is complete; if the device dries during implantation, its functionality is compromised. Unfavorable and resulting complications of poorly wetted implanted medical devices may be bio-fouling of the surface, clot formation and blood coagulation at the surface of the device among others.

The liquids can be volatile and evaporate with time. Liquids can include aqueous and non-aqueous solutions including perfluorinated liquids, fluorinated liquids, antibiotic solutions, heparinized solutions, and nutritional solutions, among others. Thus, the design and implementation of such devices pose a significant challenge.

There are two approaches to keep these implanted devices fully wetted after surgery: (a) methods and devices applied during surgery and (b) methods used to wet the devices after they have been implanted. Some, but not all, of these methods may make use of the principle of wicking, whereby a liquid is adsorbed along a surface, maintaining a fully wetted surface. Wicking is best known in porous surfaces, but also occurs along solid surfaces. Though not restricted to this method, wicking frequently works especially well when a liquid of similar chemical characteristics to the underlying solid, such as when a perfluorinated liquid is drawn along the surface of a perfluorinated solid.

### SUMMARY

Disclosed herein are wetted medical devices, methods for preparing wetted medical devices, and kits to make the same. The disclosed wetted medical devices can include a self-healing layer of lubricating fluid on a surface of the medical device. The surface includes a polymer layer having a roughened texture, which interacts with and attracts the lubricating fluid to form a lubricating fluid layer.

In some aspects, the techniques described herein relate to a medical device including: an elongate member including a first end and a second end, and a lumen passing through the elongate member from the first end to the second end; and a lubricating fluid; wherein the elongate member includes an inner surface in contact with the lumen and an outer surface; wherein the inner surface includes a porous polymer, wherein said porous polymer forms a roughened layer; wherein the lubricating fluid forms a layer on the roughened layer of the inner surface; and wherein at least a portion of the outer surface being formed from a barrier layer, the barrier layer including a polymer, wherein the polymer is impermeable to gases.

In some aspects, the techniques described herein relate to a medical device, wherein at least a portion of the outer surface includes the porous polymer, wherein said porous polymer forms a roughened layer.

In some aspects, the techniques described herein relate to a medical device, wherein the porous polymer includes PTFE, ePTFE, silicon, polyurethane, or combinations thereof.

In some aspects, the techniques described herein relate to a medical device, wherein the roughened layer of the inner and/or outer surface includes roughened features of 50 nm to 1 mm.

In some aspects, the techniques described herein relate to a medical device, wherein the lubricating fluid includes a perfluorocarbon.

In some aspects, the techniques described herein relate to a medical device, wherein the perfluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof.

In some aspects, the techniques described herein relate to a medical device, wherein the lubricating fluid further includes carbohydrates, proteins, fats, vitamins, minerals, electrolytes, and combinations thereof.

In some aspects, the techniques described herein relate to a medical device, wherein the lubricating fluid wets the roughened layer of the inner and/or outer surface and is water insoluble.

In some aspects, the techniques described herein relate to a medical device, wherein the polymer of the barrier layer includes silicone, polyurethane, or any other nonporous polymer.

In some aspects, the techniques described herein relate to a medical device, wherein the barrier layer is removable.

In some aspects, the techniques described herein relate to a medical device, further including at least one securing element that prevents fluid flow from the lumen of the elongate member.

In some aspects, the techniques described herein relate to a medical device, wherein the at least one securing element is permeable to gases.

In some aspects, the techniques described herein relate to a medical device, wherein the at least one securing element includes a means for fluid delivery.

In some aspects, the techniques described herein relate to a medical device, including one securing element disposed on the first end.

In some aspects, the techniques described herein relate to a medical device, including two securing elements disposed on the first end and the second end.

In some aspects, the techniques described herein relate to a medical device, wherein the medical device is one of catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

In some aspects, the techniques described herein relate to a medical device including: an elongate member including a first end and a second end, and a lumen passing through the elongate member from the first end to the second end; and a lubricating fluid; at least one securing element that prevents fluid flow from the lumen of the elongate member; wherein the elongate member includes an inner surface in contact with the lumen and an outer surface; wherein the inner surface includes a porous polymer, wherein said porous polymer forms a roughened layer; and wherein the lubricating fluid forms a layer on the roughened layer of the inner surface.

In some aspects, the techniques described herein relate to a medical device, wherein at least a portion of the outer surface includes the porous polymer, wherein said porous polymer forms a roughened layer.

In some aspects, the techniques described herein relate to a medical device, wherein the porous polymer includes PTFE, ePTFE, silicon, polyurethane, or combinations thereof.

In some aspects, the techniques described herein relate to a medical device, wherein the roughened layer of the inner and/or outer surface includes roughened features of 50 nm to 1 mm.

In some aspects, the techniques described herein relate to a medical device, wherein the lubricating fluid includes a perfluorocarbon.

In some aspects, the techniques described herein relate to a medical device, wherein the perfluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof.

In some aspects, the techniques described herein relate to a medical device, wherein the lubricating fluid further includes carbohydrates, proteins, fats, vitamins, minerals, electrolytes, and combinations thereof.

In some aspects, the techniques described herein relate to a medical device, wherein the lubricating fluid wets the roughened layer of the inner and/or outer surface and is water insoluble.

In some aspects, the techniques described herein relate to a medical device, wherein the at least one securing element is permeable to gases.

In some aspects, the techniques described herein relate to a medical device, wherein the at least one securing element includes a means for fluid delivery.

In some aspects, the techniques described herein relate to a medical device, including one securing element disposed on the first end.

In some aspects, the techniques described herein relate to a medical device, including two securing elements disposed on the first end and the second end.

In some aspects, the techniques described herein relate to a medical device, wherein at least a portion of the outer surface being formed from a barrier layer, the barrier layer including a polymer, wherein the polymer is impermeable to gases.

In some aspects, the techniques described herein relate to a medical device, wherein the polymer of the barrier layer includes silicone, polyurethane, or any other nonporous polymer.

In some aspects, the techniques described herein relate to a medical device, wherein the barrier layer is removable.

In some aspects, the techniques described herein relate to a medical device, wherein the medical device is one of catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

In some aspects, the techniques described herein relate to a kit including: a medical device including: an elongate member including a first end and a second end, and a lumen passing through the elongate member from the first end to the second end, the lumen having a lumen volume; and at least one securing element, wherein said at least one securing element prevents fluid flow from the lumen of the elongate member; wherein the elongate member includes an inner surface in contact with the lumen and an outer surface; wherein the inner surface includes a porous polymer, wherein said porous polymer forms a roughened layer; and a volume of lubricating fluid, wherein the volume of lubricating fluid is less than the lumen volume.

In some aspects, the techniques described herein relate to a kit, wherein at least a portion of the outer surface of the medical device includes the porous polymer, wherein said porous polymer forms a roughened layer.

In some aspects, the techniques described herein relate to a kit, wherein the porous polymer includes PTFE, ePTFE, silicon, polyurethane, or combinations thereof.

In some aspects, the techniques described herein relate to a kit, wherein the roughened layer of the inner and/or outer surface includes roughened features of 50 nm to 1 mm.

In some aspects, the techniques described herein relate to a kit, wherein the lubricating fluid includes a perfluorocarbon.

In some aspects, the techniques described herein relate to a kit, wherein the perfluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof.

In some aspects, the techniques described herein relate to a kit, wherein the lubricating fluid further includes carbohydrates, proteins, fats, vitamins, minerals, electrolytes, and combinations thereof.

In some aspects, the techniques described herein relate to a kit, wherein the lubricating fluid wets the roughened layer of the inner and/or outer surface and is water insoluble.

In some aspects, the techniques described herein relate to a kit, wherein at least a portion of the outer surface of the medical device being formed from a barrier layer, the barrier layer including a polymer, wherein the polymer is impermeable to gases.

In some aspects, the techniques described herein relate to a kit, wherein the polymer of the barrier layer includes silicone, polyurethane, or any other nonporous polymer.

In some aspects, the techniques described herein relate to a kit, wherein the barrier layer is removable.

In some aspects, the techniques described herein relate to a kit, wherein the at least one securing element is permeable to gases.

In some aspects, the techniques described herein relate to a kit, wherein the at least one securing element includes a means for fluid delivery.

In some aspects, the techniques described herein relate to a kit, wherein the kit includes one securing element.

In some aspects, the techniques described herein relate to a kit, wherein the kit includes two securing elements.

In some aspects, the techniques described herein relate to a kit, wherein the kit is used for catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

In some aspects, the techniques described herein relate to a method of preparing a medical device for insertion, wherein the medical device includes an elongate member including a first end and a second end, and a lumen passing through the elongate member from the first end to the second end, the lumen having a lumen volume; wherein the elongate member includes an inner surface in contact with the lumen and an outer surface; and wherein the inner surface includes a porous polymer, wherein said porous polymer forms a roughened layer; wherein the method includes: securing the first end with a first securing element; disposing a lubricating fluid into the lumen, thereby wetting the roughened layer of the inner surface.

In some aspects, the techniques described herein relate to a method, the method further including removing excess lubricating fluid.

In some aspects, the techniques described herein relate to a method, the method further including securing the second end with a second securing element.

In some aspects, the techniques described herein relate to a method, the method further including removing the first securing element.

In some aspects, the techniques described herein relate to a method, wherein at least a portion of the outer surface being formed from a barrier layer, the barrier layer including a polymer, wherein the polymer is impermeable to gases.

In some aspects, the techniques described herein relate to a method, wherein the barrier layer includes silicone, polyurethane, or any other nonporous polymer.

In some aspects, the techniques described herein relate to a method, wherein the barrier layer is removed before insertion.

In some aspects, the techniques described herein relate to a method, wherein at least a portion of the outer surface includes the porous polymer, wherein said porous polymer forms a roughened layer.

In some aspects, the techniques described herein relate to a method, wherein the porous polymer includes PTFE, ePTFE, silicon, polyurethane, or combinations thereof.

In some aspects, the techniques described herein relate to a method, wherein the roughened layer of the inner and/or outer surface includes roughened features of 50 nm to 1 mm.

In some aspects, the techniques described herein relate to a method, wherein the lubricating fluid includes a perfluorocarbon.

In some aspects, the techniques described herein relate to a method, wherein the perfluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof.

In some aspects, the techniques described herein relate to a method, wherein the lubricating fluid further includes carbohydrates, proteins, fats, vitamins, minerals, electrolytes, and combinations thereof.

In some aspects, the techniques described herein relate to a method, wherein the first securing element is permeable to gases.

In some aspects, the techniques described herein relate to a method, wherein the first securing element includes a means for fluid delivery.

In some aspects, the techniques described herein relate to a method, wherein the medical device is one of catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

### BRIEF DESCRIPTION OF THE DRAWINGS

FIG. 1A shows an example of a tube of a medical device with one clamp, securing fluid inside of the tube.

FIG. 1B shows an example of a tube of a medical device with two clamps, securing fluid inside of the tube.

FIG. 2 shows an example of a tube of a medical device with a peal-off layer to preserve wet devices during implant.

FIG. 3 shows an example of a permanent sleeve or coating covering a wetted tube of a medical device.

FIG. 4 shows an example of a reservoir external to the device which allows the implanted device to continually refresh.

FIG. 5 shows an example of an additional porous material attached to the outside of a porous tube of a medical device.

FIGS. 6A-6C show examples of hydrocephalus shunt implantation device.

FIGS. 7A-7B show examples of hydrocephalus shunt implantation device.

FIG. 8 shows an example of a vascular catheter implantation device.

FIG. 9 shows an example use of a ventricular catheter.

FIG. 10 shows an example use of a ventricular catheter.

FIG. 11 shows an example use of a ventricular catheter.

FIG. 12 shows an example of a medical device including an elongate member.

### DETAILED SPECIFICATION

During implantation, the implanted device can remain wet through a combination of methods and lubricating compositions.

Disclosed herein is a wetted medical device, methods for preparing wetted medical devices, and kits to make the same. The disclosed wetted medical device includes a self-healing layer of lubricating fluid on a surface of the medical device. The surface includes a polymer layer having a roughened texture, which interacts with and attracts the lubricating fluid to form a lubricating fluid layer.

In an exemplary aspect, the medical device includes an elongate member, the elongate member being the feature of the device that must remain wet throughout implantation and any subsequent medical procedure. The elongate member includes a porous polymer surface having features such as a roughened surface. The roughened surface may have features that are on the scale of 50 nm to 100 microns, 50 nm to 500 microns, 500 nm to 1 mm, or 500 microns to 1 mm. The roughened surface promotes the lubricating fluid to form a film, which may be stable on the time scale of relevant medical uses.

In some aspects, as shown in FIG. 12, the medical device includes an elongate member (1200) including a first end (1202) and a second end (1203), a lumen (1204) passing through the elongate member (1200) from the first end (1202) to the second end (1203), and a lubricating fluid. The elongate member includes an inner surface in contact with the lumen and an outer surface. The inner surface includes a porous polymer, wherein said porous polymer forms a roughened layer. The lubricating fluid forms a layer on the inner surface of the elongate member. The elongate member may include at least a portion of the outer surface being formed from a barrier layer (1205), the barrier layer (1205) including a polymer, wherein the polymer is impermeable to gases.

In some aspects, the elongate member can further comprise one or more additional lumens passing through the elongate member from the first end to the second end. The one or more additional lumens can be fluidly independent from the first lumen. Alternatively, the one or more additional lumens can be fluidly connected to the first lumen.

In some aspects, the elongate member may have a linear, branched, or multiple parallel elongate sub-members, whereby the elongate member includes a plurality of ends and a plurality of lumens. It is contemplated that the features of the disclosure herein are not limited to one configuration of an elongate member of the medical device.

In some aspects, at least a portion of the outer surface includes the porous polymer, wherein said porous polymer forms a roughened layer, whereby the lubricating fluid forms a layer on the roughened layer.

In some aspects, the porous polymer includes PTFE, ePTFE, silicon, polyurethane, or combinations thereof. The roughened layer includes porous features of 50 nm to 1 mm.

In some aspects, the lubricating fluid includes a perfluorocarbon, wherein the perfluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof. The lubricating fluid may further include carbohydrates, proteins, fats, vitamins, minerals, electrolytes, and combinations thereof.

In some aspects, the lubricating fluid wets the roughened layer of the inner and/or outer surface and is water insoluble.

In some aspects, the polymer of the barrier layer includes silicone, polyurethane, or any other nonporous polymer. The barrier layer is configured to be removed, so that during the use of the medical device, the barrier layer may be removed.

Referring now to FIG. 2, showing the barrier layer is a peel-away layer (201) on the outside of an elongate member or tubular device (200).

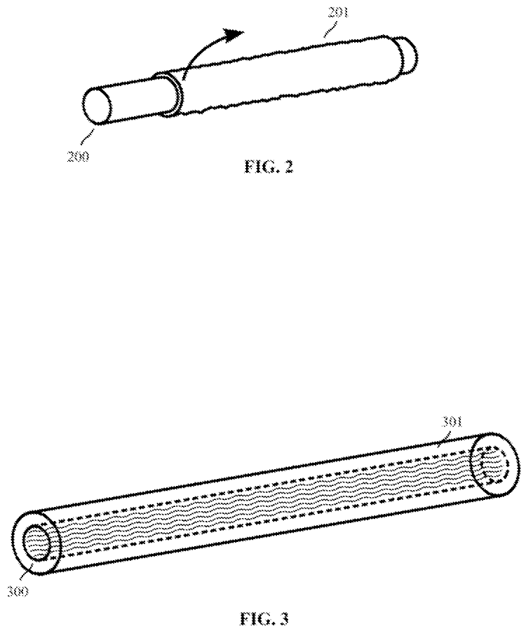

Referring now to FIG. 3, an elongate member, in this example shown as a tubular device (300), including a barrier layer, is shown as a permanently affixed sleeve (301).

In some aspects, the medical device may further include at least one securing element that prevents fluid flow from the lumen of the elongate member. The at least one securing element is permeable to gases and may include a means for fluid delivery such as a luer lock.

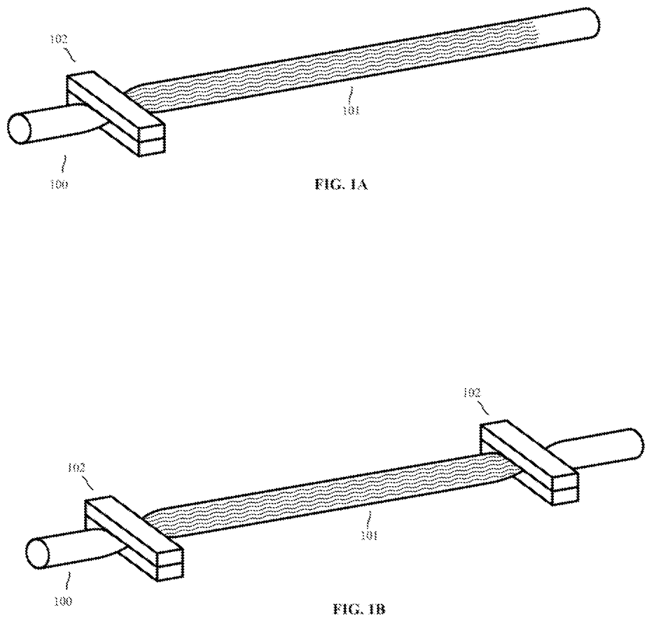

In some aspects, one securing element disposed on the first end. In other aspects, two securing elements disposed on the first end and the second end. Referring now to FIG. 1A, the securing element is a clamp (102) at the first end of the elongate member, which may be a tubular device (100). Referring now to FIG. 1B, the two securing elements are clamp (102) at the first and second ends of the elongate member, which may be a tubular device (100).

In some aspects, the medical device is one of catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

In some aspects, the medical device includes an elongate member including a first end and a second end, a lumen passing through the elongate member from the first end to the second end, a lubricating fluid, and at least one securing element that prevents fluid flow from the lumen of the elongate member. Referring now to FIGS. 1A and 1B, for example, that show the at least one securing member, which is a clam (102) in this example, preventing liquid (101) entrapped in the elongate member, in this example, a tubular device (100).

The elongate member includes an inner surface in contact with the lumen and an outer surface. The inner surface includes a porous polymer, wherein said porous polymer forms a roughened layer. The lubricating fluid forms a layer on the roughened layer of the inner surface. In preferred embodiments, the lubricating fluid is stable on the roughened surface in biological environments for an extended period of time, for instance, up to 10 minutes, up to 30 minutes, up to 1 hour, up to 2 hours, or up to 3 hours.

In some aspects, at least a portion of the outer surface may include the porous polymer, wherein said porous polymer forms a roughened layer, whereby the lubricating fluid forms a layer on the roughened layer.

In some aspects, the porous polymer includes PTFE, ePTFE, silicon, polyurethane, or combinations thereof. The porous features of the roughened layer are on the order of 50 nm to 1 mm.

In some aspects, the lubricating fluid includes a perfluorocarbon, wherein the perfluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof. The lubricating fluid may further include carbohydrates, proteins, fats, vitamins, minerals, electrolytes, and combinations thereof. The lubricating fluid wets the roughened layer of the inner and/or outer surface and is water insoluble.

In some aspects, the at least one securing element is permeable to gases and includes a means for fluid delivery, such as a luer lock.

In some aspects, one securing element disposed on the first end. In other aspects, two securing elements disposed on the first end and the second end. Referring now to FIGS. 1A and 1B, for example, that show the at least one securing member, which is a clam (102) in this example, preventing liquid (101) entrapped in the elongate member, in this example, a tubular device (100).

In some aspects, at least a portion of the outer surface includes a barrier layer. The barrier layer includes a polymer, wherein the polymer is impermeable to gases. The polymer of the barrier layer includes silicone, polyurethane, or any other nonporous polymer. In exemplary embodiments, the barrier layer is removable. Referring now to FIG. 12, the elongate member (1200) is shown with a barrier layer (1205).

In some aspects, the medical device is one of catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

Disclosed herein is a kit for wetting a medical device. The kit includes a medical device comprising an elongate member comprising a first end and a second end, a lumen passing through the elongate member from the first end to the second end, the lumen having a lumen volume, and at least one securing element, wherein said at least one securing element prevents fluid flow from the lumen of the elongate member. The elongate member includes an inner surface in contact with the lumen and an outer surface. The inner surface includes a porous polymer, wherein said porous polymer forms a roughened layer. The kit further includes a volume of lubricating fluid, wherein the volume of lubricating fluid is less than the lumen volume.

In some aspects, at least a portion of the outer surface may include the porous polymer, wherein said porous polymer forms a roughened layer. The porous polymer includes PTFE, ePTFE, silicon, polyurethane, or combinations thereof. The porous features of the roughened layer may be on the order of 50 nm to 1 mm.

In some aspects, the lubricating fluid includes a perfluorocarbon, wherein the perfluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof. The lubricating fluid may further include carbohydrates, proteins, fats, vitamins, minerals, electrolytes, and combinations thereof. The lubricating fluid wets the roughened layer of the inner surface and is water insoluble.

The elongate member may include at least a portion of the outer surface being formed from a barrier layer, the barrier layer including a polymer, wherein the polymer is impermeable to gases. The polymer of the barrier layer includes silicone, polyurethane, or any other nonporous polymer. In some aspects, the barrier layer is removable from the elongate member.

Referring now to FIG. 2, an elongate member, in this example shown as a tubular device (200) including a barrier layer that is a peel-away layer (201) on the outside of an elongate member or tubular device (200).

Referring now to FIG. 3, an elongate member, in this example shown as a tubular

device (300), including a barrier layer, is shown as a permanently affixed sleeve (301).

In some aspects, the at least one securing element is permeable to gases and includes a means for fluid delivery, such as a luer lock.

In some aspects, the kit includes one securing element, whereas in other aspects, the kit includes two securing elements.

In some aspects, the medical device in the kit is used for catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

In an exemplary aspects, a method of preparing a medical device for insertion is described. The medical device may include an elongate member including a first end and a second end, a lumen passing through the elongate member from the first end to the second end, the lumen having a lumen volume. The elongate member having an inner surface and an outer surface, wherein the inner surface includes a porous polymer. The porous polymer forming a roughened layer.

The method includes securing the first end with a first securing element and disposing a lubricating fluid into the lumen, thereby wetting the roughened layer of the inner surface. The method may further include removing excess lubricating fluid. In some aspects, the method further includes securing the second end with a second securing element. The method further includes removing the first securing element.

In some aspects, at least a portion of the outer surface includes a barrier layer, the barrier layer being a polymer, wherein the polymer is impermeable to gases and includes silicone, polyurethane, or any other nonporous polymer. In some aspects, the method includes removing the barrier layer is removed before insertion.

In some aspects, the medical device is one of catheters, central line catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

In one aspect, when the implanted device is a tube, the tube is filled or partially filled with a lubricating liquid and the lubricating liquid is trapped within the device. The lubricating liquid may be trapped through a temporary clamping device towards each end of the device. The clamping device can be removed just prior to completing the surgery allowing it to function as intended. Alternatively, the lubricating liquid may be trapped by various functional plugs at either end, in which case, one or more plugs may be present at the time of implantation. One or more plugs may be gas or air permeable, while a second plug may be a port through which the lubricating liquid can be injected.

The entrapped fluid keeps the inside of the device wet during a surgical procedure. If the device is porous, the entrapped fluid will continue to wet both the inside and outside of the device. Extra fluid can be removed from the device via a hypodermic needle (e.g. through a port or through the body of the device or other process just prior to removing the clamps if this is not preferred. The device can be manufactured to be clamped on either end.

In some instances, the device may comprise a porous material and only require wetting on the inside, such as a catheter, stunt, or graft. In this instance, the outside of the device is coated with a non-porous coating or covering. Such a covering could include any solid which prevents the lubricating liquid from evaporating through the outside of the device. Such coverings could include silicone, polyurethane, or any other polymer which prevents liquids from migrating from the inside to the outside of a porous device. Said coating may be a permanent fixture to the inserted device or it may be removable.

In some instances, the inside and outside surfaces of the device may require wetting. In such cases, a non-porous coating is removed after a time period of the surgical procedure, this type of removeable covering is hereafter referred to as a “peel-away” cover (per FIG. 2). The peel-away cover prevents the liquid from evaporating during the procedure. This peel-away cover remains in place for a portion of the device where the device extends outside of the body. The covering outside of the body would remain while the device is in use to prevent evaporation of the liquid from the device into the environment outside of the patient. Examples of such uses where the portion of peel-away cover may remain outside of the body after the device is implanted include External Ventricular Drains, urinary catheters, catheters used in percutaneous vascular procedures, and vascular catheters.

In other implementations, a locking solution of the lubricant liquid can be used for catheters and other implanted devices which extend outside of the body. Locking solutions are liquids which are inserted into a catheter between uses and are frequently removed prior to use.

Directly pouring liquid onto the device once it is implanted and still exposed prior to closing up the patient.

In another aspect, the implanted device is wetted or re-wetted following insertion.

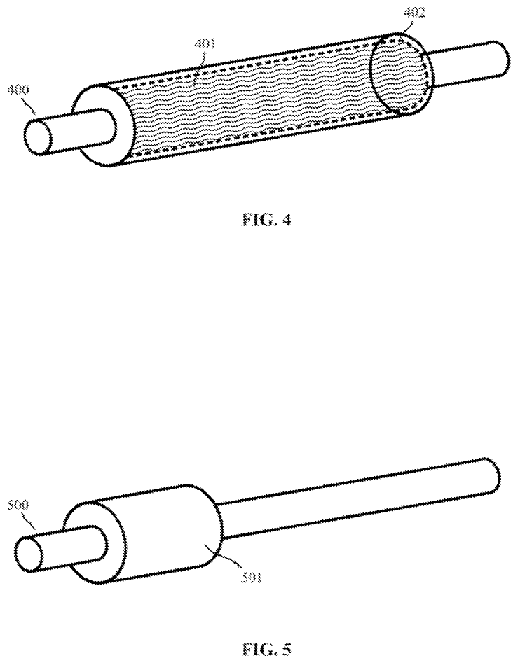

In one implementation, a pouch can be wrapped around all or a portion of the device. The pouch can be filled with the liquid which can constantly refresh the liquid on the outside of the device. Referring now to FIG. 4, which shows a tubular device (400) encircled by a reservois (402) of the liquid (401). If the device is porous, the liquid in the pouch will wick throughout the material, wetting the entire device. The pouch can be made from a material either identical to, similar to, or completely different from the porous material of the device. The pouch must contain the lubricating liquid and hold it against the device, such that the liquid can wick into the bulk material of the device. The liquid can be re-injected into the pouch at some time interval after the device is implanted into the patient to restore the liquid in the pouch.

In another implementation, a porous coating can be wrapped around all or a portion of the device, as shown in FIG. 5. The porous coating can be filled with the liquid which can constantly re-wet the outside of the device. If the device is porous, the liquid in the coating will wick throughout the material, wetting the entire device. The liquid can be re-injected into the coating after the device is implanted into the patient to restore the liquid in the porous. In some implementations, the non-porous coating may be a thicker portion of the porous material of the device included on the outer surface of the device. This thicker, porous portion will (a) act as a reservoir of the liquid, and (b) provide a location for additional liquid to be injected (into the porous material) to refresh the liquid on the device.

The pouch, sleeve, thicker portion, or bulk of the device itself discussed above could be refilled with the liquid after the implanted device is in the patient through an injection through the skin directly into the pouch, sleeve, bulky portion, or device itself. The liquid then wicks from the pouch to the device.

If the device has an air-facing side, aerosolized or vaporized liquid can be blown against surfaces, rewetting the surface as the aerosolized droplets land on the surface or vaporized liquid condenses on the surface. An example of this mode of refreshing the surface is hollow fiber used in extracorporeal membrane oxygenation (ECMO) systems. Hollow fiber may be made of polypropylene, polymethyl pentene, expanded PTFE, porous PVDF. Membrane can be fluorinated to enhance the LP permeance.

Such replenishment device may have a condenser to capture excess or vaporized LP and recirculate with the sweep gas flow.

Such replenishment methods also apply to extracorporeal devices. Extracorporeal devices such as extracorporeal membrane oxygenation which require hollow fiber bundles and blood perfusion tubing, the hollow fiber can be continually replenished by either (a) aerosolizing or vaporizing the fluid into the air-side flow of the oxygenator, allowing it to condense or land on the internal surfaces of the hollow fiber or (b) create a reservoir of liquid on one end of the fibers which the fibers run through. The reservoir of fluid will wick along the fibers as they pass through the blood chamber.

In aspects of the method, the inner and/or outer surface of the device comprises a rough surface such that the liquid wets the surface. The liquid may be a fluorinated and/or perfluorinated liquid. A chosen liquid is required to have a suitable vapor pressure, in some instances, a liquid with a higher vapor pressure is preferred so that evaporation is sufficiently slow for the intended application on a device and surgical procedure.

In the present disclosure the fluorinated and/or perfluorinated liquid used may include, but is not limited to, one or more liquids selected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof.

Liquid nutrient solutions may include, but are not limited to carbohydrates, proteins, fats, vitamins, minerals and electrolytes.

The device may include, but are not exclusive of the following: catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, cerebral stents.

Some example devices and methods of implantation are described in the following section.

### EXAMPLES

Example 1: Fluid clamped in implanted medical device. Clamps (102) can be placed towards the ends of a tubular device (100) entrapping liquid (101) in the device while it is being implanted. Once the implantation is nearly done, the clamps (102) can be removed. Optionally, the liquid (101) may be withdrawn from the device via a syringe prior to removal of the clamps. The clamps (102) could be removed from the device and patient prior to the end of the procedure or a permanent fixture on the device which remains unclamped but in place for future clamping and device refill with liquid (101).

Example 2: Peal-off layer to preserve wet device during implant. A previously wetted device (200) can have a peal-away layer (201) which can be removed either during or after the procedure to implant the device, retaining the wet layer on the device during implantation. The peal-away layer (201) may have a variety of patterns of perforations which allow this layer to be torn off in a manner suitable to the procedure. A portion of this peel-away layer may remain intact after the device is implanted if a portion of the device remains outside of the patient. Such devices could include external ventricular catheters, vascular catheters, urinary catheters, and catheters used for vascular procedures.

Example 3: Sleeve or coating covering the wetting device. A permanent sleeve or covering (301) may cover all or a portion of the wetted device (300) which covers the implanted device during implantation. This cover may be of a dissimilar material which is impervious or significantly less pervious than that of the device (300), maintaining the inside of the device (300) wet. If the device is tubular in shape, even though it has open ends, significantly less liquid will evaporate through the ends than would from the exposed external surface of the device. An example of such a sleeve is a silicone coating on an ePTFE (porous) device. The silicone coating on the outside of the ePTFE device would reduce the evaporation of a liquid infused into the ePTFE. The sleeve could also be a separately manufactured silicone sleeve which slides snugly over the ePTFE device.

Example 4: A reservoir external to the device which allows the implanted device to continually refresh. A reservoir (401) external to the device (400) which holds liquid (402) against the outside of the device, allowing it to replenish any liquid which may leave the device through its use in the body. This reservoir may be replenished by injecting additional liquid into it using a device such as a hypodermic needle. Other methods to replenish the reservoir include but are not limited to a port which can be accessed through the skin external to the reservoir (401). The port would channel newly infused liquid into the reservoir. The reservoir (401) can be a wide range of sizes, both along the diameter of the device and in thickness outside of the device.

Example 5: Additional porous material being attached to the outside of a porous implanted medical device. The reservoirs discussed in FIG. 4 need not only be created through voids, but also may be created by adding additional porous material (501) to a device (500) to create a porous reservoir of material. The additional porous material (501) may be of any thickness or length along the device (500) but must be in direct contact with the outside of the device (500) to allow the liquid in the additional material to wick into the bulk of the device. After the device (500) has been implanted, additional liquid may be injected into the bulk of the extra porous material (501) attached to the device (500) to provide new liquid to keep the device wetted.

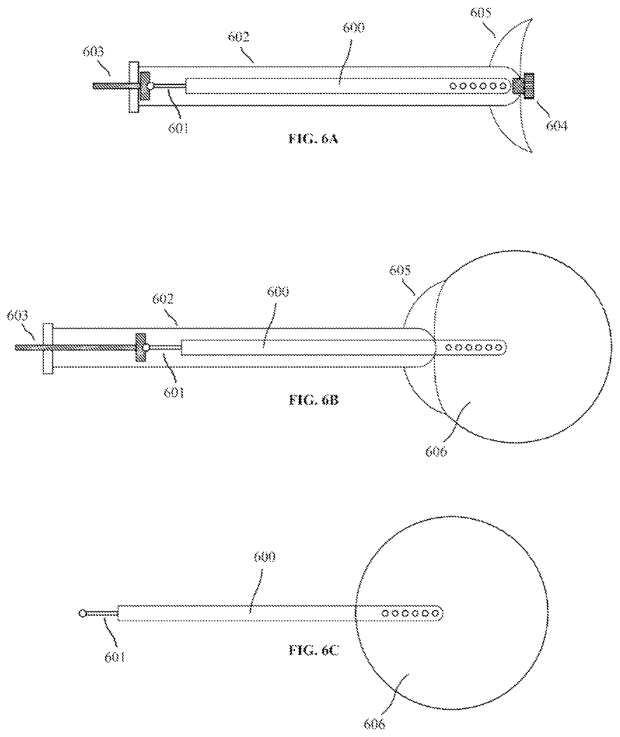

Example 6: Hydrocephalus Shunt Implantation Device. FIGS. 6A-6C illustrate an example wetted ventricular catheter. As shown in FIG. 6A, a wetted ventricular catheter (600) fitted on stylet (601) is packaged inside the sterile barrier (602). The assembly can be made available at the point of care in a breathable pouch.

Sterile barrier (602) can be a hard plastic tube fitted with a plunger (603) at the distal end and a removable cap (604) at the proximal end.

Sterile barrier (602) can have a covering flap (605) around the removable cap present at the proximal end. In some embodiments, the covering flap (605) can be affixed to the sterile barrier with an adhesive.

The covering flap can be made of silicone or other plastic film to cover the insertion site. As shown in FIG. 6B, during implantation, the removable cap is opened. The covering flap (605) can cover a region surrounding the insertion site on the cranium (606) to allow this delivery device to dock. In some embodiments, the covering flap (605) can be coated with an antimicrobial agent (e.g., an antibiotic) to reduce the risk of infection associated with implantation. In some embodiments, the covering flap (605) can include an adhesive to adhere the covering flap to the cranium (606).

Plunger (603) can be depressed to actuate the stylet catheter assembly in vivo. The sterile barrier (602) can then be removed after implantation.

This design can facilitate catheter implantation without exposure to the air. Additional infection resistance can be conferred by the inclusion of an antimicrobial coating on the covering flap (605).

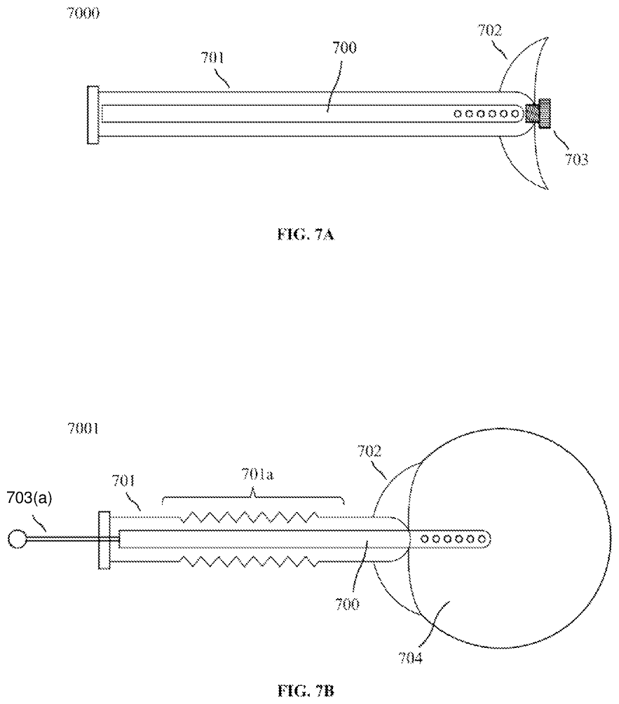

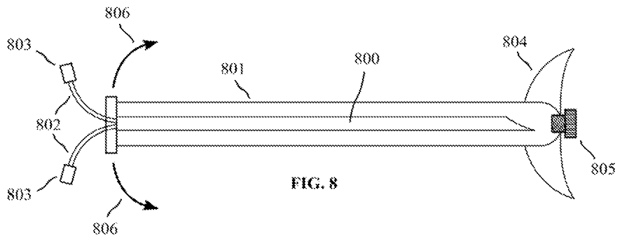

Example 7: Hydrocephalus Shunt Implantation Device. FIGS. 7A-7B illustrate an example wetted ventricular catheter. As shown in FIG. 7A, a wetted ventricular catheter (700) is packaged inside the sterile barrier (701). The assembly can be made available at the point of care in a breathable pouch. A stylet (703a) can be provided in the same or a separate packaging, but outside of the sterile barrier.

Sterile barrier (701) can be a collapsible tube (e.g., a collapsible plastic tube) and/or have a peel away feature. Sterile barrier (702) can have an insertion port at the distal end (not labeled) and a removable cap (703) at the proximal end. The insertion port at the distal end can allow stylet (703a) insertion while catheter is still in the sterile packaging.

Sterile barrier (701) can have a covering flap (702) around the removable cap present at the proximal end. In some embodiments, the covering flap (702) can be affixed to the sterile barrier with an adhesive.

The covering flap can be made of silicone or other plastic film to cover the insertion site. As shown in FIG. 7B, during implantation, the removable cap is opened. Stylet (703a) is inserted into the catheter lumen. The covering flap (702) can cover a region surrounding the insertion site on the cranium (704) to allow this delivery device to dock. In some embodiments, the covering flap (702) can be coated with an antimicrobial agent (e.g., an antibiotic) to reduce the risk of infection associated with implantation. In some embodiments, the covering flap (702) can include an adhesive to adhere the covering flap to the cranium (704).

When stylet is pushed in the sterile barrier wall collapses as the catheter is deployed. The sterile barrier (701) can then be removed after implantation.

This design can facilitate catheter implantation without exposure to the air. Additional infection resistance can be conferred by the inclusion of an antimicrobial coating on the covering flap (702).

Example 8: vascular catheter implantation device. FIG. 8 illustrates an example wetted ventricular catheter. As shown in FIG. 8, a wetted vascular catheter (800) is packaged inside the sterile barrier (801). The assembly can be made available at the point of care in a breathable pouch. The sterile barrier (801) can extend from the distal tip up to a bifurcation (802).

Sterile barrier (801) can be a collapsible tube (e.g., a collapsible plastic tube) and/or have a peel away feature. Sterile barrier can have a removable cap (805) at the distal end.

Sterile barrier (801) can have a covering flap (804) around the removable cap present at the distal end. In some embodiments, the covering flap (702) can be affixed to the sterile barrier with an adhesive.

The covering flap can be made of silicone or other plastic film to cover the insertion site. During implantation, the removable cap (805) can be opened. A guidewire can be inserted into the catheter lumen and used to advance to the catheter. The covering flap (804) can cover a region surrounding the insertion site on the cranium to allow this delivery device to dock. In some embodiments, the covering flap (804) can be coated with an antimicrobial agent (e.g., an antibiotic) to reduce the risk of infection associated with implantation. In some embodiments, the covering flap (804) can include an adhesive to adhere the covering flap to the cranium.

When the catheter is advanced, the sterile barrier (801) can be peeled away (as shown by arrows (806). This design can facilitate catheter implantation without exposure to the air. Additional infection resistance can be conferred by the inclusion of an antimicrobial coating on the covering flap (804).

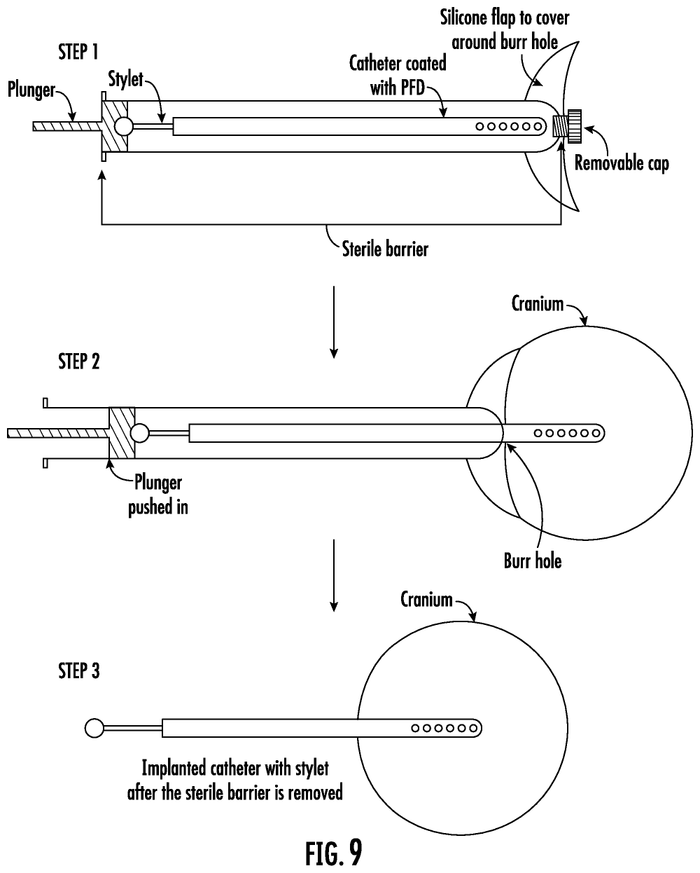

Example 9: Example use of a Ventricular Catheter. In FIG. 9, a wetted ventricular catheter fitted in stylet is packaged inside the sterile barrier and make available at the point of care in a breathable pouch. The sterile barrier is a hard plastic tube fitted with a plunger at the distal end and a removable cap at the proximal end. The sterile barrier has a silicone flap around the removable cap present at the proximal end.

During implantation, the removable cap is opened. The silicone flap (could be antibiotic coated) covers around the insertion site to allow this delivery device to dock. The plunger is pushed in to move the stylet catheter assembly in vivo. Then, the sterile barrier is removed after implantation.

This device allows catheter implantation without exposing it to the air and has additional infection resistance out of antibiotic coating of the silicone flap.

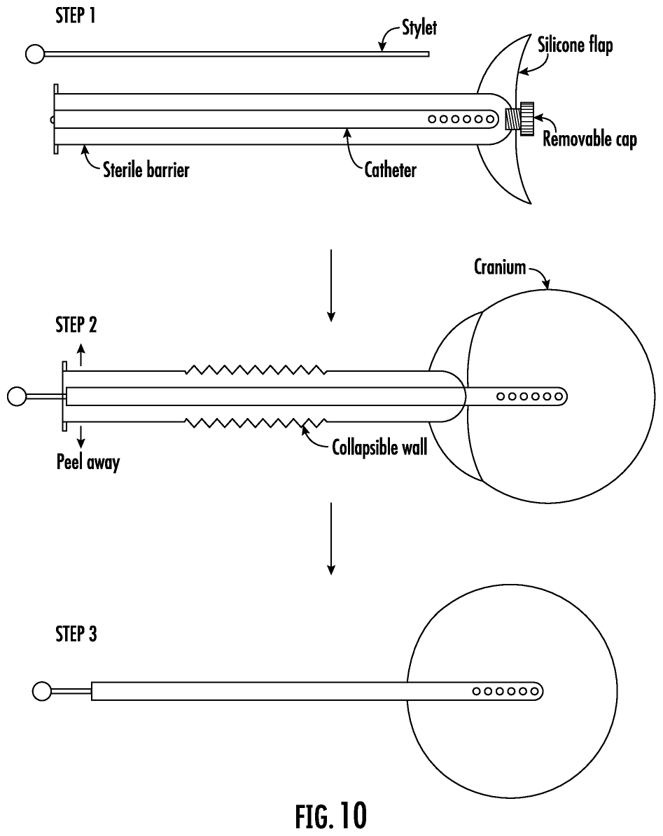

Example 10: Example use of a Ventricular Catheter. In FIG. 10, a wetted ventricular catheter is packaged inside the sterile barrier and made available at the point of care in a breathable pouch. The stylet is placed outside this sterile barrier but inside the breathable pouch. The sterile barrier is a collapsible plastic tube and/or may have a peel away feature. The sterile barrier has an insertion port at the distal end and a removable cap at the proximal end. The insertion port at the distal end allows stylet insertion while the catheter is still in the sterile packaging. The sterile barrier has a silicone flap around the removable cap present at the proximal end.

During implantation, the removable cap is opened. The stylet is inserted into the catheter lumen, then the silicone flap (could be antibiotic coated) covers around the insertion site to allow this delivery device to dock. When the stylet is pushed in, the sterile barrier wall collapses and allows sterile barrier to peel away after implantation.

This insertion device allows catheter implantation without exposing it to the air and provides additional infection resistance out of antibiotic coating of the silicone flap.

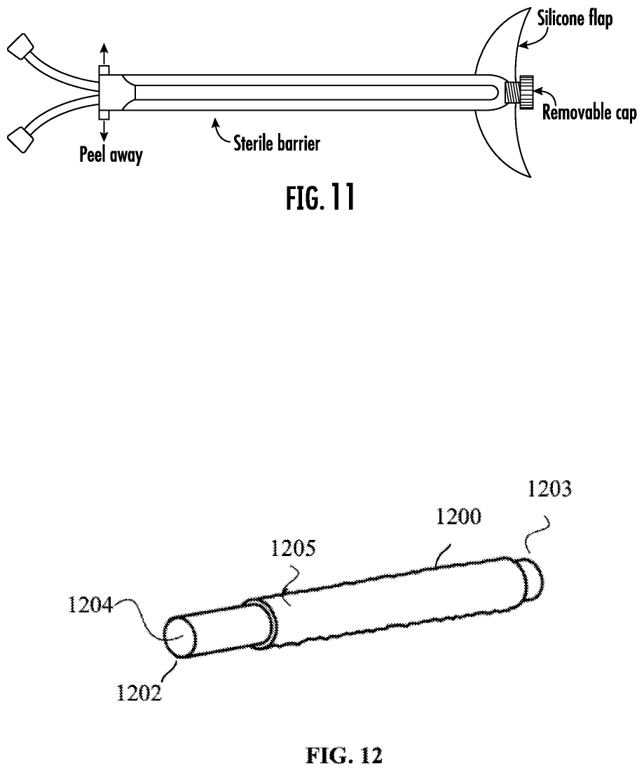

Example 11: Example use of a Ventricular Catheter. FIG. 11 shows a wetted vascular catheter that is packaged inside the sterile barrier and made available at the point of care in a breathable pouch. The sterile barrier covers from the distal tip up to the bifurcation. The sterile barrier is a collapsible plastic tube and/or may have a peel away feature. The sterile barrier has a removable cap at the distal end. The sterile barrier has a silicone flap around the removable cap present at the distal end.

During implantation, the removable cap is opened, and the guidewire is inserted into the catheter lumen. The silicone flap (could be antibiotic coated) covers the insertion site to allow this delivery device to dock. When the catheter is pushed in, the sterile barrier wall collapses and or allows sterile barrier to peel away.

This insertion device allows catheter implantation without exposing it to the air and provides additional infection resistance out of antibiotic coating of the silicone flap.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The expressions “ambient temperature” and “room temperature” as used herein are understood in the art and refer generally to a temperature from about 20° C. to about 35° C.

As used herein, the term “composition” is intended to encompass a product comprising the specified ingredients in the specified amounts, as well as any product which results, directly or indirectly, from a combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example embodiments.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then about 1% by weight, e.g., less than about 0.5% by weight, less than about 0.1% by weight, less than about 0.05% by weight, or less than about 0.01% by weight of the stated material, based on the total weight of the composition.

As used herein, the terms “substantially identical reference composition,” “substantially identical reference article,” or “substantially identical reference electrochemical cell” refer to a reference composition, article, or electrochemical cell comprising substantially identical components in the absence of an inventive component. In another exemplary aspect, the term “substantially,” in, for example, the context “substantially identical reference composition,” or “substantially identical reference article,” or “substantially identical reference electrochemical cell” refers to a reference composition, article, or an electrochemical cell comprising substantially identical components and wherein an inventive component is substituted with a common in the art component.

The devices, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods, in addition to those shown and described herein, are intended to fall within the scope of the appended claims. Further, while only certain representative devices, systems, and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less; however, other combinations of steps, elements, components, and constituents are included, even though not explicitly stated.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove and that many modifications and other embodiments are intended to be included within the scope of the appended claims. Moreover, although specific terms are employed herein, as well as in the claims which follow, they are used only in a generic and descriptive sense and not for the purposes of limiting the described invention nor the claims which follow.

Unless defined otherwise, all technical and scientific terms used herein have the same meanings as commonly understood by one of skill in the art to which the disclosed invention belongs. Publications cited herein and the materials for which they are cited are specifically incorporated by reference.

While aspects can be described and claimed in a particular statutory class, such as the system statutory class, this is for convenience only and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

In view of the described processes and compositions, hereinbelow are described certain more particularly described aspects of the inventions. These particularly recited aspects should not, however, be interpreted to have any limiting effect on any different claims containing different or more general teachings described herein, or that the “particular” aspects are somehow limited in some way other than the inherent meanings of the language and formulas literally used therein.

The present invention may be understood more readily by reference to the following detailed description of various aspects of the invention and the examples included therein and to the Figures and their previous and following description.

What is claimed is:

1. A medical device comprising:
an elongate member comprising a first end and a second end , and a lumen passing through the elongate member from the first end to the second end; and
a lubricating fluid;
wherein the elongate member comprises an inner surface in contact with the lumen and an outer surface;
wherein the inner surface comprises a porous polymer, wherein said porous polymer forms a roughened layer;
wherein the lubricating fluid forms a layer on the roughened layer of the inner surface; and
wherein at least a portion of the outer surface being formed from a barrier layer, the barrier layer comprising a polymer, wherein the polymer is impermeable to gases.

2. The medical device of claim 1, wherein at least a portion of the outer surface comprises the porous polymer, wherein said porous polymer forms a roughened layer, and wherein the lubricating fluid forms a layer on the roughened layer of the outer surface.

3. The medical device of claim 1, wherein the porous polymer comprises PTFE, ePTFE, silicon, polyurethane, or combinations thereof.

4. The medical device of claim 2, wherein the roughened layer of the inner and/or outer surface comprises roughened features of 50 nm to 1 mm.

5. The medical device of claim 1, wherein the lubricating fluid comprises a perfluorocarbon.

6. The medical device of claim 5, wherein the perfluorocarbon comprises perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof.

7. The medical device of claim 1, wherein the lubricating fluid further comprises carbohydrates, proteins, fats, vitamins, minerals, electrolytes, and combinations thereof.

8. The medical device of claim 1, wherein the lubricating fluid wets the roughened layer of the inner surface and is water insoluble.

9. The medical device of claim 1, wherein the polymer of the barrier layer comprises silicone, polyurethane, or any other nonporous polymer.

10. The medical device of claim 1, wherein the barrier layer is removable.

11. The medical device of claim 1, further comprising at least one securing element that prevents fluid flow from the lumen of the elongate member.

12. The medical device of claim 11, wherein the at least one securing element is permeable to gases.

13. The medical device of claim 11, wherein the at least one securing element comprises a means for fluid delivery.

14. The medical device of claim 11, comprising one securing element disposed on the first end.

15. The medical device of claim 11, comprising two securing elements disposed on the first end and the second end.

16. The medical device of claim 1, wherein the medical device is one of catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

17. A medical device comprising:
an elongate member comprising a first end and a second end, and a lumen passing through the elongate member from the first end to the second end; and
a lubricating fluid;
at least one securing element that prevents fluid flow from the lumen of the elongate member;
wherein the elongate member comprises an inner surface in contact with the lumen and an outer surface;
wherein the inner surface comprises a porous polymer, wherein said porous polymer forms a roughened layer; and
wherein the lubricating fluid forms a layer on the roughened layer of the inner surface.

18. The medical device of claim 17, wherein at least a portion of the outer surface comprises the porous polymer, wherein said porous polymer forms a roughened layer, and wherein the lubricating fluid forms a layer on the roughened layer of the outer surface.

19. The medical device of claim 17, wherein the porous polymer comprises PTFE, ePTFE, silicon, polyurethane, or combinations thereof. 20 The medical device of claim 18, wherein the roughened layer of the inner and/or outer surface comprises roughened features of 50 nm to 1 mm.

21. The medical device of claim 17, wherein the lubricating fluid comprises a perfluorocarbon.

22. The medical device of claim 21, wherein the perfluorocarbon comprises perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™M), 2,2,2-trifluoroethanol, silicone solutions, and combinations thereof.

23. The medical device of claim 17, wherein the lubricating fluid further comprises carbohydrates, proteins, fats, vitamins, minerals, electrolytes, and combinations thereof.

24. The medical device of claim 17, wherein the lubricating fluid wets the roughened layer of the inner and/or outer surface and is water insoluble.

25. The medical device of claim 17, wherein the at least one securing element is permeable to gases.

26. The medical device of claim 17, wherein the at least one securing element comprises a means for fluid delivery.

27. The medical device of claim 17, comprising one securing element disposed on the first end.

28. The medical device of claim 17, comprising two securing elements disposed on the first end and the second end.

29. The medical device of claim 17, wherein at least a portion of the outer surface being formed from a barrier layer, the barrier layer comprising a polymer, wherein the polymer is impermeable to gases.

30. The medical device of claim 29, wherein the polymer of the barrier layer comprises silicone, polyurethane, or any other nonporous polymer.

31. The medical device of claim 29, wherein the barrier layer is removable.

32. The medical device of claim 17, wherein the medical device is one of catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

33. A kit comprising:
a medical device comprising: an elongate member comprising a first end and a second end, and a lumen passing through the elongate member from the first end to the second end, the lumen having a lumen volume; and
at least one securing element, wherein said at least one securing element prevents fluid flow from the lumen of the elongate member;
wherein the elongate member comprises an inner surface in contact with the lumen and an outer surface;
wherein the inner surface comprises a porous polymer, wherein said porous polymer forms a roughened layer; and
a volume of lubricating fluid, wherein the volume of lubricating fluid is less than the lumen volume.

34. The kit of claim 33, wherein at least a portion of the outer surface comprises the porous polymer, wherein said porous polymer forms a roughened layer.

35. The kit of claim 33, wherein the porous polymer comprises PTFE, ePTFE, silicon, polyurethane, or combinations thereof.

36. The kit of claim 34, wherein the roughened layer of the inner and/or outer surface comprises roughened features of 50 nm to 1 mm.

37. The kit of claim 33, wherein the lubricating fluid comprises a perfluorocarbon.

38. The kit of claim 37, wherein the perfluorocarbon comprises perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof.

39. The kit of claim 33, wherein the lubricating fluid further comprises carbohydrates, proteins, fats, vitamins, minerals, electrolytes, and combinations thereof.

40. The kit of claim 33, wherein the lubricating fluid wets the roughened layer of the inner and/or outer surface and is water insoluble.

41. The kit of claim 33, wherein at least a portion of the outer surface being formed from a barrier layer, the barrier layer comprising a polymer, wherein the polymer is impermeable to gases.

42. The kit of claim 41, wherein the polymer of the barrier layer comprises silicone, polyurethane, or any other nonporous polymer.

43. The kit of claim 41, wherein the barrier layer is removable.

44. The kit of claim 33, wherein the at least one securing element is permeable to gases.

45. The kit of claim 33, wherein the at least one securing element comprises a means for fluid delivery.

46. The kit of claim 33, wherein the kit comprises one securing element.

47. The kit of claim 33, wherein the kit comprises two securing elements.

48. The kit of claim 33, wherein the kit is used for catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.

49. A method of preparing a medical device for insertion, wherein the medical device comprises an elongate member comprising a first end and a second end, and a lumen passing through the elongate member from the first end to the second end, the lumen having a lumen volume;
wherein the elongate member comprises an inner surface in contact with the lumen and an outer surface; and
wherein the inner surface comprises a porous polymer, wherein said porous polymer forms a roughened layer;
wherein the method comprises:
securing the first end with a first securing element;
disposing a lubricating fluid into the lumen, thereby wetting the roughened layer of the inner surface.

50. The method of claim 49, the method further comprising removing excess lubricating fluid.

51. The method of claim 50, the method further comprising securing the second end with a second securing element.

52. The method of claim 50, the method further comprising removing the first securing element.

53. The method of claim 49, wherein at least a portion of the outer surface being formed from a barrier layer, the barrier layer comprising a polymer, wherein the polymer is impermeable to gases.

54. The method of claim 53, wherein the barrier layer comprises silicone, polyurethane, or any other nonporous polymer.

55. The method of claim 53, wherein the barrier layer is removed before insertion.

56. The method of claim 49, wherein at least a portion of the outer surface comprises the porous polymer, wherein said porous polymer forms a roughened layer.

57. The method of claim 49, wherein the porous polymer comprises PTFE, ePTFE, silicon, polyurethane, or combinations thereof.

58. The method of claim 49, wherein the roughened layer of the inner and/or outer surface comprises roughened features of 50 nm to 1 mm.

59. The method of claim 49, wherein the lubricating fluid comprises a perfluorocarbon.

60. The method of claim 59, wherein the perfluorocarbon comprises perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134a™, HFA227ea™, methyl perfluorobutylether, methyl perfluoropropyl ether (3M Novec 7000™), 2,2,2-trifluoroethanol, silicone solutions, and combinations thereof.

61. The method of claim 49, wherein the lubricating fluid further comprises carbohydrates, proteins, fats, vitamins, minerals, electrolytes, and combinations thereof.

62. The method of claim 49, wherein the first securing element is permeable to gases.

63. The method of claim 49, wherein the first securing element comprises a means for fluid delivery.

64. The method of claim 49, wherein the medical device is one of catheters, central line catheters, hemodialysis catheters, peripherally inserted central line catheters, peripheral catheters, shunts, vascular grafts, arteriovenous grafts, vascular ports, urinary catheters, urinary stents, angioplasty catheters, defibrillator, pacemaker, left ventricular assist device, breast implants, cochlear implants, orthopedic implants, hydrocephalus shunts, external ventricular drain, vascular stents, coronary stents, or cerebral stents.
